(19) United States
(12) Patent Application Publication
(10) Pub. No.: US 2023/0265129 A1
(43) Pub. Date: Aug. 24, 2023

(54) Subunit Vaccine Composition For African Swine Fever, And Preparation Therefor And Use Thereof

(71) Applicant: NOVO BIOTECH CORP, Shaoxing (CN)

(72) Inventors: Qiang ZHANG, Shaoxing, Zhejiang (CN); Hong QIAN, Shaoxing, Zhejiang (CN); Youqiang WU, Shaoxing, Zhejiang (CN); Sufang WU, Shaoxing, Zhejiang (CN); Ying CHE, Shaoxing, Zhejiang (CN)

(21) Appl. No.: 18/005,367

(22) PCT Filed: Jul. 15, 2021
(86) PCT No.: PCT/CN2021/106375

§ 371 Date: Jan. 13, 2023

(30) Foreign Application Priority Data
Jul. 15, 2020 (CN) 202010683211.5
Jul. 2, 2021 (CN) 202110752873.8

(51) Int. Cl. C07K 14/01; A61K 39/12; C12N 7/00; A61P 31/20

(52) U.S. Cl. CPC C07K 14/01; A61K 39/12; C12N 7/00; A61P 31/20; A61K 2039/552

(57) ABSTRACT

The present invention provides a subunit vaccine composition for African swine fever, and a preparation therefor and use thereof, which fall within the technical field of animal vaccines and veterinary biological products. The vaccine comprises an exterior envelope protein CD2V derived from African swine fever virus and an exterior envelope capsid protein p72 derived from African swine fever virus and a pharmaceutically acceptable adjuvant. The method for preparing the vaccine comprises: 1) preparing the exterior envelope protein CD2V derived from African swine fever virus and the exterior envelope capsid protein p72 derived from African swine fever virus; 2) mixing the exterior envelope protein CD2V derived from African swine fever virus with the exterior envelope capsid protein p72 derived from African swine fever virus prepared in step 1), so as to prepare an antigen solution; and 3) emulsifying the antigen solution and ISA 201 VG at a volume ratio of 46:54.

### CROSS-REFERENCE TO RELATED APPLICATIONS

This application is a national stage application of the Patent Cooperation Treaty (PCT) international application titled “Subunit Vaccine Composition For African Swine Fever, And Preparation Therefor And Use Thereof”, international application number PCT/CN2021/106375, filed in the China National Intellectual Property Administration (CNIPA) on Jul. 15, 2021. PCT international application number PCT/CN2021/106375 claims priority to and benefit of Chinese Patent Application No. 202010683211.5, filed on Jul. 15, 2020, and Chinese Patent Application No. 202110752873.8, filed on Jul. 2, 2021. The specifications of the above referenced patent applications are incorporated herein by reference in their entirety.

### FIELD OF THE INVENTION

The present invention relates to a subunit vaccine composition for African swine fever, and preparation therefor and use thereof, and belongs to the technical field of animal vaccines and veterinary biological products.

### BACKGROUND

African swine fever (ASF) is an acute, febrile and highly contagious disease of pigs caused by African swine fever virus (ASFV). Pigs infected with African swine fever virus are clinically characterized by skin congestion, visceral bleeding, and high fever. The incidence rate and mortality rate are as high as 100%. The disease has been classified as class A disease by the World Organization for Animal Health. China is a big country of livestock and poultry breeding, wherein the amount of pig breeding accounts for 56.6% of the total amount of pig breeding in the world, pork consumption accounts for 49.6% of the world pork consumption. China is the world's second largest producer of poultry meat after the United State. The African swine fever plague has spread to the whole country of China since the first African swine fever plague in Liaoning Province of China on Aug. 4, 2018. This has caused a devastating blow to Chinese pig breeding. Therefore, it is urgent to develop a vaccine with strong immunogenicity, good safety and low cost.

ASFV is the only member of the African swine fever virus genus (Asfivirus) of the African swine fever virus family (Asfarviridae), and is the only known arbovirus with DNA genome at present. The viral genome is a linear, covalently closed double stranded DNA (dsDNA) molecule. The specificity and length of ASFV isolates in different regions were different, and the length of different isolates is about 170-190 kb. At present, there is no effective vaccine to prevent and control the disease at home and abroad.

African swine fever has a complex structure. ASFV contains 151-167 open reading frames (ORFs), encoding 150-200 proteins. Mature virus particles contain more than 50 structural proteins. The structure of African swine fever virus analyzed at present shows that the average particle size of African swine fever virus is 260-300 nm. Recombinant 3D modeling shows that African swine fever has five layers of structure, the outermost layer is the exterior envelope containing the capsule membrane, the fourth layer is the capsid protein, the third layer is also the inner envelope containing the capsule membrane, the second layer is the nucleocapsid, and the innermost layer is the nuclear blast (N. Wang et al., Science 10.1126/science. aaz1439 (2019)). The complexity and variability of the virus makes the production of a vaccine against ASFV infection complicated. Wherein, CD2V protein is the only protein located in the exterior envelope, p72 is the main protein of the outer capsid, and its protein content accounts for about 30% of the total protein content of virions (Alejo A, et al., J Virol 10.1128/JVI.01293-18 (2018)).

Prior art has reported several vaccines based on expressions of CD2V or p72 proteins. For example, Argilaguet J M et al. constructed a BacMam-sHAPQ based on baculovirus vector, which can induce specific T cell response after immunizing pigs.

Some pigs can resist the attack of homologous sublethal virus strains, and a large number of IFN-γ secreting T cells were detected in pig blood after 17 days of challenge (Argilaguet J M, et al. Antiviral Res. 10.1016/j.antiviral.2013.02.005). In 2016, Lokhandwala S et al. recombined A151R, B119L, B602L, EP402RΔPRR, B438L, K205R and A104R into adenovirus vector respectively, and, it would elicit strong antigen-specific IgG response to African swine fever and IFN-γ after immunizing pigs with adjuvant in recombinant adenovirus (Lokhandwala S, et al. PLoS One. 2017; 12(5): e0177007). In 2017, Loperamadrid J et al. screened five ASFV antigens using the Vaxign system. Using 293 (HEK) cells of human embryonic kidney and three MVA carrier antigens expresses p72, p54, p12 antigens and three MVA vector antigens (B646L, EP153R, and EP402R), using primary immunization enhances immunity. Inoculation with HEK purified ASFV protein promoted humoral immune response, but cellular immunity is weak. MVA vector antigen can promote cell immunity to produce IFN-γ. However, the results of challenge protection were not reported. In 2020, Lynette C et al. use adenovirus and poxvirus as vectors to express eight genes including B602L, B646L (p72), CP204L (P30), E183L (P54), E199L, EP153R, F317L, MGF505-5R. It can provide pigs with complete protection against African swine fever at a high immune dose. However, there are still infections according to the data, that is, there are still great side effects. Therefore, the safety and effectiveness are still not enough, and there are many genes to be expressed. Thus it is not conducive to large-scale production and application.

The above-mentioned reports either contain the exterior envelope CD2V protein or the outer capsid p72 protein. But it was not reported whether the correct folding is the correct trimer structure. Therefore, the current reported vaccines have not been reported to effectively and safely protect immunized pigs against African swine fever. This is because the African swine fever virus has a complex structure with multi-layer structure, and it is difficult for a single component to play a safe and effective protection.

### SUMMARY OF THE INVENTION

The technical problems to be solved by the present invention is firstly to provide a subunit vaccine composition with strong immunogenicity, good safety, and fundamentally purifying African swine fever; secondly to overcome the viral variation caused by nucleic acid recombination of live vaccine with the genome of natural African swine fever virus, thereby solve great biosafety risks and possible immune interference problems; thirdly to provide a method for preparing subunit vaccine composition for African swine fever and its application in the prevention of African swine fever.

On the basis of screening a large number of proteins, the inventors accidentally found that the preparation of subunit vaccine composition containing CD2V protein and p72 protein containing trimer can provide complete and safe protection for pigs, and lay the foundation for the development of subunit vaccines, and has important significance for the prevention of African swine fever virus.

The inventors of the present invention believe that the exterior envelope protein and capsid protein have the infectivity ability respectively, so it is necessary to combine the two antigens to effectively provide protection. In addition, it was also found that the structure of exterior envelope protein and capsid protein was particularly critical, and the exterior envelope protein should be sugar modified protein; the outer capsid protein p72 should be correctly expressed and folded, and a certain amount of triploid virus like particles are required. If it cannot be correctly expressed and folded, it will cause reaction and aggravate the development of the disease.

The present invention provides a subunit vaccine composition for African swine fever, wherein the vaccine composition comprises African swine fever virus exterior envelope protein CD2V and African swine fever virus outer capsid protein p72 and pharmaceutically acceptable adjuvant; wherein the African swine fever virus exterior envelope protein CD2V is a glycosylated protein expressed in the eukaryotic system, the African swine fever virus outer capsid protein p72 is a trimeric protein.

In the technical solution of the subunit vaccine composition for African swine fever of the present invention, preferably, the content of the trimeric protein is not less than 40% of the total amount of the exterior envelope subunit protein p72 derived from African swine fever virus. More preferably, the exterior envelope subunit protein CD2V derived from African swine fever virus is mixed with the exterior envelope subunit protein p72 derived from African swine fever virus in equal mass ratio.

In the technical solution of the subunit vaccine composition for African swine fever of the present invention, the concentration of the exterior envelope subunit protein CD2V derived from African swine fever virus and the exterior envelope subunit protein p72 derived from African swine fever virus in the vaccine composition are 25 μg/vaccine-200 μg/vaccine respectively. More preferably, the concentration of the exterior envelope subunit protein CD2V derived from African swine fever virus and the exterior envelope subunit protein p72 derived from African swine fever virus in the vaccine are 50 μg/vaccine respectively.

In the technical solution of the subunit vaccine composition for African swine fever of the present invention, the pharmaceutically acceptable adjuvant may be water adjuvant such as aluminum gel adjuvant, Montanide GEL 01 PR adjuvant, etc., also may be oil adjuvant such as white oil, ISA 206 VG, etc., and more preferably, the adjuvant is ISA 201 VG.

In the technical solution of the subunit vaccine composition for African swine fever of the present invention, the preservative may be a mercuric preservative. Preferably, the preservative is thiomersalate, and the content of thiomersalate in each vaccine is less than 0.01%. More preferably, the content of thiomersalate is 2 μg/vaccine.

According to another aspect of the present invention, the present invention further provides a method for preparing the subunit vaccine composition for African swine fever, comprising the following steps: 1) preparing the exterior envelope subunit protein CD2V derived from African swine fever virus and the exterior envelope subunit protein p72 derived from African swine fever virus; wherein, the exterior envelope subunit protein CD2V derived from African swine fever virus is a glycosylated protein expressed in the eukaryotic system; the exterior envelope subunit protein p72 derived from African swine fever virus is a trimeric protein, and the content of the trimeric protein is not less than 40% of the total amount of the exterior envelope subunit protein p72 derived from African swine fever virus; 2) mixing the exterior envelope subunit protein CD2V derived from African swine fever virus with the exterior envelope subunit protein p72 derived from African swine fever virus prepared by step 1) to obtain an antigen solution; wherein, mixing the exterior envelope subunit protein CD2V derived from African swine fever virus with the exterior envelope subunit protein p72 derived from African swine fever virus in a mass ratio of 1-8:8-1; and 3) mixing the antigen solution with the ISA 201 VG in a volume ratio of 46:54 and then emulsifying to obtain a vaccine composition.

In the technical solution of the preparation method of the present invention, the exterior envelope subunit protein CD2V derived from African swine fever virus is mixed with the exterior envelope subunit protein p72 derived from African swine fever virus in equal mass ratio.

In the technical solution of the preparation method of the present invention, preferably, the antigen solution of the vaccine composition further comprises preservatives.

According to another aspect of the present invention, the present invention further provides an application of the subunit vaccine composition for African swine fever in preparation of a vaccine for preventing African swine fever virus infection.

It has been found from the animal immunization examples of the present invention that the subunit vaccine composition of African swine fever can effectively resist the attack of African swine fever virus, and has good protective effects on immunized pigs.

In comparison with the prior art, the present invention firstly explicitly proposes the subunit vaccine composition for African swine fever, its preparation method and application. The vaccine composition of the present invention has the advantages of strong immunogenicity, good safety, no immune interference and fundamentally purifying ASFV. Moreover, the vaccine composition can not only effectively prevent and protect pigs from the infection of African swine fever virus, but also eliminate hidden dangers of virus recombination and mutation, after performing immunization with the vaccine.

### BRIEF DESCRIPTION OF DRAWINGS

FIG. 1 shows the molecular sieve results of the African swine fever virus subunit protein p72 after purification.

FIG. 2 shows the temperature changes of different vaccines after being challenged by the African swine fever virus.

FIG. 3 shows the survivals of different vaccines after being challenged by the African swine fever virus.

### DETAILED DESCRIPTION OF THE INVENTION

### Example 1: Preparation of the Exterior Envelope Subunit Protein CD2V Derived from African Swine Fever Virus and the Exterior Envelope Subunit Protein p72 Derived from African Swine Fever Virus

1.1 Refer to the preparation method of African swine fever exterior envelope subunit protein CD2V of Application No. 201910004596.5 or 201910069838.9 of the applicant, or the preparation method of the exterior envelope subunit protein CD2V derived from African swine fever virus of other patents or documents (prokaryotic expression system expression, such as CHO, 293T cells, etc.).

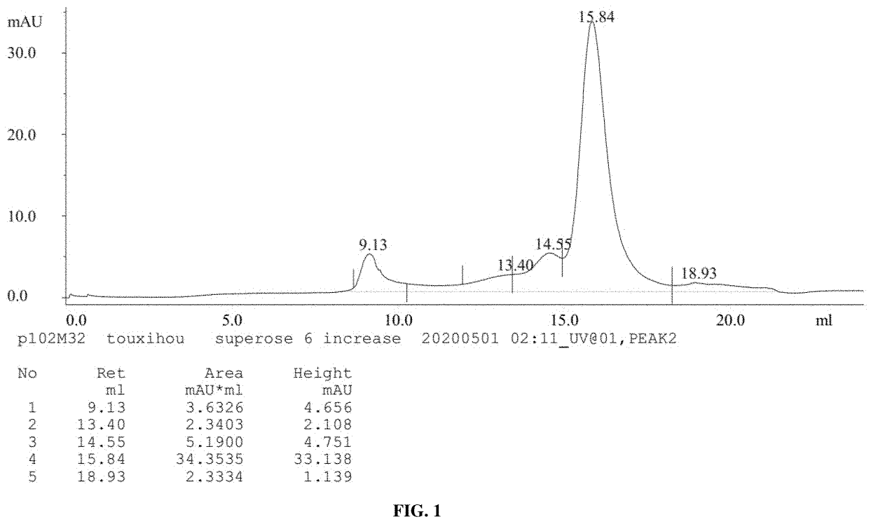

1.2 Reference document is Structure of the African swine fever virus major capsid protein written by Liu Q, Ma B, Qian N, et al., p72. Cell Res. 2019; 29 (11): 953-955. Or other protein expression modes (such as the eukaryotic expression system, the insect baculovirus expression system, the CHO cell expression system, etc). Or the preparation method of African swine fever virus subunit protein p72 of other patents or documents. The content of the trimer reaches more than 40%. As shown in FIG. 1, it may be seen from the comparison between the peaking results of protein p72 molecular sieve and standard column chromatogram that the peak volume of Peak 1 is 9.13 ml, its molecular weight is greater than 660 kDa, which is a p72 protein polymers; the peak volume of Peak 2 is 13.40 ml, the peak volume of Peak 3 is 14.55 ml, and its molecular weight is between 440 kDa and 660 kDa, which is p72 protein oligomer; the peak volume of Peak 4 is 15.84 ml, and its molecular weight is between 158 kDa and 440 kDa, which is a p72 protein trimer (the molecular weight of p102M32 protein trimer is about 232 kDa); the peak volume of Peak 5 is 18.93 ml, its molecular weight is between 6.5 kDa and 13.7 kDa, which may be a mixed protein in the purified protein.

It may be seen from FIG. 1 that the area (34.3535) of Peak 4 accounts for 71.79% of the total area (47.8497). It indicates that 71.79% of the purified p102M32 protein is trimer before further optimization of the buffer system. It meets predictive analysis.

### Example 2: Preparation of African Swine Fever Virus Subunit Vaccines (the Preparation of 2 ml/Head, a Total of 200 ml, is Taken as an Example

The supplies and materials used to prepare the vaccine are pre-sterilized in advance. The preparation process is completed in a biosafety cabinet or other instrument or environment to ensure the sterilization of the whole preparation process.

1. Preparation of ISA 201 VG: According to a volume ratio of the antigen solution to the adjuvant of 46:54, take 108 ml of the adjuvant volume and add the adjuvant to a prepared reagent bottle in advance, and then seal, and place in a 33° C. water bath pot and then preheat for 30 min.

2. According to a volume ratio of the antigen solution to the adjuvant of 46:54, the total volume of the water phase is 92 ml. Calculate the volume of the exterior envelope subunit protein CD2V derived from African swine fever virus and the exterior envelope subunit protein p72 derived from African swine fever virus respectively, according to the concentration of the exterior envelope subunit protein CD2V derived from African swine fever virus and the exterior envelope subunit protein p72 derived from African swine fever virus and the total content of protein in the vaccine. If the preservative thiomersalate is added to the antigen solution, calculate the volume of the thiomersalate according to the original concentration of thiomersalate and the content of thiomersalate in the vaccine. The total volume of the antigen solution is replenished to 92 ml with PBS buffer solution or other buffer solutions, and then uniformly mixed and preheated in a water bath pot at 33° C. for 30 min. For example, the concentration of the exterior envelope subunit protein CD2V derived from African swine fever virus and the exterior envelope subunit protein p72 derived from African swine fever virus is 0.5 mg/ml respectively, the original concentration of the thiomersalate is 30 mg/ml, and the content of the exterior envelope subunit protein CD2V derived from African swine fever virus and the exterior envelope subunit protein p72 derived from African swine fever virus in the vaccine is 50 μg/vaccine (2 ml/vaccine) respectively, and the content of the thiomersalate in the vaccine is 2 μg/ml. The particular configuration is shown in Table 1 below.

TABLE 1

| Title | Volume | |
|---|---|---|
| CD2V | 10 | ml |
| P72 | 10 | ml |
| thiomersalate | 13.3 | μl |
| PBS buffer solution | 72 | ml |
| total volume | 92 | ml |

3. Stirring: Add the adjuvant preheated to a beaker prepared, adjust a height and speed of a mixer, and then quickly add the antigen solution preheated to the oil phase, and then continue to stir for 10-20 minutes. Generally, choose a stirring speed and a stirring time according to the preparation volume, for example, generally select 350 rpm/min and stir for 10 min if preparing 200 ml of the vaccine composition.

4. Stabilization: Place the vaccine stirred of Step 3 in a water bath pot at 20° C. and stand for 1 h, and then place in a refrigerator at 4° C. overnight.

5. Sub-package: The vaccines stabilized are sub-packaged and labeled as need.

### Example 3: Immune Challenge Test of the African Swine Fever Subunit Vaccines of CD2V and p72

3.1 Preparation of the Vaccines

Prepare the protein and the vaccines according to the methods of Examples 1 and 2. The particular vaccines are shown in Table 2 below:

TABLE 2

| name | CD2V | p72 | Thiomersal |
|---|---|---|---|
| vaccine 1 | 100 μg/vaccine | 100 μg/vaccine | 2 μg/ml |
| | | (p72 monomer) | |
| vaccine 2 | 100 μg/vaccine | 100 μg/vaccine, | 2 μg/ml |
| | | the monomer content | |
| | | of p72 is 20% | |
| vaccine 3 | 100 μg/vaccine | 100 μg/vaccine, | 2 μg/ml |
| | | the trimer content | |
| | | of p72 is 40% | |
| vaccine 4 | 100 μg/vaccine | 100 μg/vaccine, | 2 μg/ml |
| | | the trimer content | |
| | | of p72 is 71.79% | |

3.2 Immunological Experiment

Twenty-five piglets at 35-40 days of age (the African swine fever virus negative) are screened, and randomly divided into five groups, and each group has five piglets. One group served as a blank control group, the other four groups served as an immunization group, vaccine 1 to vaccine 4 are immunized respectively. The blank control group is intramuscularly injected with 2 ml of normal saline each time, and the other four immunization groups are intramuscularly injected with 2 ml of corresponding vaccine each time. The immunization is strengthened once after three weeks of preliminary immunization. Attack virus after three weeks of secondary immunization.

3.3 Challenge Protection Test

Perform an intramuscular injection to the neck with 10 HAD ASFV viruses after three weeks of immunization for the experimental groups immunized with vaccine 1-4 of Step 3.2 and the blank control group. Each group is fed in isolation, and then continuously observe for 21 days (ASFV belongs to a major animal epidemic disease, and this part of the experiment is conducted in the P3 laboratory of Chinese center for disease control and prevention according to biosafety requirements).

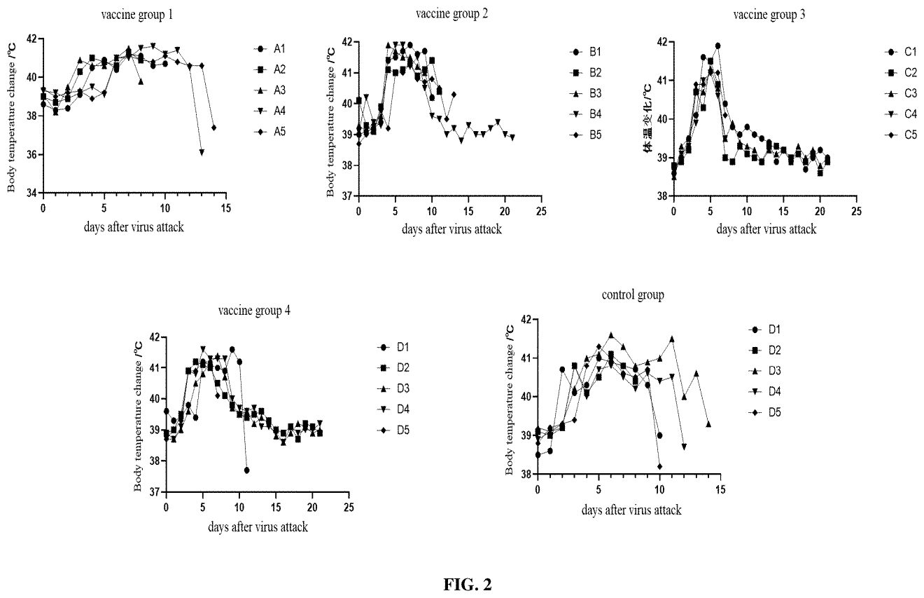

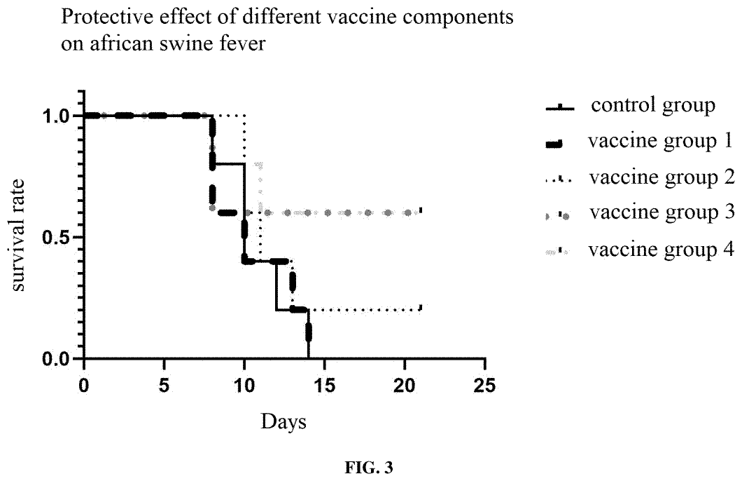

Check regularly body temperature every morning after challenge (i.e., attacking virus). The results are shown in FIG. 2 and FIG. 3. The body temperature began to rise suddenly and the food intake decreased 3-6 days later after immunization against African swine fever virus in all groups. In Vaccine Group 1, pigs A2 and A3 died on the 8th day, and all pigs died on the 14th day. In Vaccine Group 2, pigs B1 and B3 died suddenly on the 10th day after challenge, pigs B2 died on the 12th days after challenge, pigs B4 had a temperature rise on the 4th day after challenge, and then recovered to normal on the 10th day after challenge, and survived until the end of the test; pigs B5 died on the 13th days after challenge. In Vaccine Group 3, the body temperature of pigs C4 and C5 began to rise on the third day after challenge, pigs C4 and C5 died on the 8th day after challenge, and the temperature and the food intake of the other three pigs gradually recovered to normal on the 8th day after challenge, and survived until the end of the test. In Vaccine Group 4, pigs D5 died on 8th days after challenge, pigs D1 died on the 11th day after challenge, and the temperature and the food intake of the other three pigs gradually recovered to normal on the 9th day after challenge, and survived at the end of the test on the 21st day.

No typical symptoms of African swine fever such as skin cyanosis, bleeding are observed in all pigs of the Vaccine Groups 3 and 4. Part of pigs in Vaccine Group 2 had skin cyanosis symptoms. One survived pig had no typical symptoms of African swine fever such as skin cyanosis and bleeding. In Vaccine Group 1 and the control group, all pigs died on the 14th day after challenge, and some pigs appear cyanosis, blood in stool and other symptoms. All pigs are culled under observation for 21 days. It can be seen that the content of p72 trimer is crucial to the final protection effects. The combined immunization of p72 and CD2V with content of higher than 40% trimer can provide 60% protection. The particular vaccine test results are shown in Table 3 below.

TABLE 3

| | | | protective |
|---|---|---|---|
| Name | CD2V | p72 | rate |
| vaccine 1 | 100 μg/vaccine | 100 μg/vaccine | 0 |
| | | (p72 monomer) | |
| vaccine 2 | 100 μg/vaccine | 100 μg/vaccine, | 20% |
| | | the trimer content of | |
| | | p72 is 20% | |
| vaccine 3 | 100 μg/vaccine | 100 μg/vaccine, | 60% |
| | | the trimer content of | |
| | | p72 is 40% | |
| vaccine 4 | 100 μg/vaccine | 100 μg/vaccine, | 60% |
| | | the trimer content of | |
| | | p72 is 71.79% | |
| control | | | 0 |
| group | | | |

The present invention is exemplified by the above examples. However, the present invention is not limited by the above-mentioned examples. These specific examples and embodiments are intended to help those skilled in the art to implement the present invention. Any person skilled in this field can easily make further improvements and refinements without deviating from the spirit and scope of the present invention. The present invention is therefore limited only by the content and scope of the claims of the present invention, with the intent to cover all alternatives and equivalents included in the spirit and scope of the present invention defined by the appended claims.

We claim:

1. A subunit vaccine composition for African swine fever, wherein the vaccine composition comprises an exterior envelope subunit protein CD2V derived from African swine fever virus, an exterior envelope subunit protein p72 derived from African swine fever virus, and pharmaceutically acceptable adjuvant; wherein the exterior envelope subunit protein CD2V derived from African swine fever virus is a glycosylated protein expressed in the eukaryotic system, the exterior envelope subunit protein p72 derived from African swine fever virus is a trimeric protein.

2. The subunit vaccine composition for African swine fever according to claim 1, wherein the content of the trimeric protein is not less than 40% of the total amount of the exterior envelope subunit protein p72 derived from African swine fever virus.

3. The subunit vaccine composition for African swine fever according to claim 1, wherein the exterior envelope subunit protein CD2V derived from African swine fever virus is a glycosylated protein with antigenicity, and is at least a glycosylated protein expressed in the eukaryotic system selected from the group consisting of full-length proteins, fusion proteins fused with chaperone protein, truncated proteins, fusion proteins fused with chaperone protein and protein after truncated.

4. The subunit vaccine composition for African swine fever according to claim 1, wherein the exterior envelope subunit protein CD2V derived from African swine fever virus is mixed with the exterior envelope subunit protein p72 derived from African swine fever virus in a mass ratio of 1-8:8-1.

5. The subunit vaccine composition for African swine fever according to claim 4, wherein the exterior envelope subunit protein CD2V derived from African swine fever virus is mixed with the exterior envelope subunit protein p72 derived from African swine fever virus in equal mass ratio.

6. The subunit vaccine composition for African swine fever according to claim 5, wherein the concentration of the exterior envelope subunit protein CD2V derived from African swine fever virus and the exterior envelope subunit protein p72 derived from African swine fever virus are 25 μg/vaccine-200 μg/vaccine respectively.

7. The subunit vaccine composition for African swine fever according to claim 6, wherein the concentration of the exterior envelope subunit protein CD2V derived from African swine fever virus and the exterior envelope subunit protein p72 derived from African swine fever virus are 50 μg/vaccine respectively.

8. The subunit vaccine composition for African swine fever according to claim 1, wherein the pharmaceutically acceptable adjuvant is ISA 201 VG.

9. The subunit vaccine composition for African swine fever according to claim 1, wherein the vaccine composition further comprises an immunopotentiator and/or preservative.

10. The subunit vaccine composition for African swine fever according to claim 9, wherein the immunopotentiator is GM-CSF, the concentration of the GM-CSF is 20 μg/vaccine-60 μg/vaccine, preferably 40 μg/vaccine.

11. The subunit vaccine composition for African swine fever according to claim 10, wherein the preservative is thiomersalate, and the content of the thiomersalate is 2 μg/vaccine.

12. A method for preparing the vaccine composition according to claim 1, comprising the following steps:
1) preparing the exterior envelope subunit protein CD2V derived from African swine fever virus and the exterior envelope subunit protein p72 derived from African swine fever virus; wherein, the exterior envelope subunit protein CD2V derived from African swine fever virus is a glycosylated protein expressed in the eukaryotic system; the exterior envelope subunit protein p72 derived from African swine fever virus is a trimeric protein, and the content of the trimeric protein is not less than 40% of the total amount of the exterior envelope subunit protein p72 derived from African swine fever virus;
2) mixing the exterior envelope subunit protein CD2V derived from African swine fever virus with the exterior envelope subunit protein p72 derived from African swine fever virus prepared by step 1) to obtain an antigen solution; wherein, mixing the exterior envelope subunit protein CD2V derived from African swine fever virus with the exterior envelope subunit protein p72 derived from African swine fever virus in a mass ratio of 1-8:8-1; and
3) mixing the antigen solution with the ISA 201 VG in a volume ratio of 46:54 and then emulsifying to obtain a vaccine composition.

13. The method according to claim 12, wherein mixing the exterior envelope subunit protein CD2V derived from African swine fever virus with the exterior envelope subunit protein p72 derived from African swine fever virus in equal mass ratio.

14. The method according to claim 12, wherein in step 2), the antigen solution further comprises a preservative.

15. An application of the subunit vaccine composition for African swine fever according to claim 1 in preparation of a vaccine for preventing African swine fever virus infection.
